(19) United States
(12) Patent Application Publication
(10) Pub. No.: US 2006/0135412 A1
(43) Pub. Date: Jun. 22, 2006

(54) Methods for the treatment of alzheimers disease and compositions therefore

(76) Inventors: Dalia Cohen, Livingston, NJ (US); Larry Alexander Gaither, Bradford, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/535,385

(22) PCT Filed: Nov. 21, 2003
(86) PCT No.: PCT/EP03/13115

§ 371 Date: Dec. 13, 2005

Related U.S. Application Data
(60) Provisional application No. 60/428,623, filed on Nov. 22, 2002.
(60) Provisional application No. 60/471,138, filed on May 16, 2003.

(51) Int. Cl. A61K 38/17; A61K 38/12; A61K 48/00

(52) U.S. Cl. 514/9; 514/12; 514/44

(57) ABSTRACT

The invention discloses previously unknown modifiers of Aβ secretion. These proteins are identified as suitable targets for the development of new therapeutics to treat, prevent or ameliorate pathological conditions associated with Aβ secretion, including Alzheimer's Disease. The invention also relates to methods to treat, prevent or ameliorate said pathological conditions and pharmaceutical compositions therefore comprising modulators with inhibitory effects on the activity and/or expression of these modifiers.

### FIELD OF THE INVENTION

The present invention relates to methods for the treatment, prevention or amelioration of pathological conditions associated with Aβ secretion including, but not limited to, Alzheimer's Disease.

### BACKGROUND OF THE INVENTION

Alzheimer's disease (AD) is characterized by the extracellular accumulation of amyloid plaques in the brain composed of the 40 or 42 amino acid Aβ peptide. This extracellular accumulation of the Aβ42 peptide is the hallmark pathology of the disease state and therefore thought to be the most important player in the cause of AD. While another common lesion of the AD brain is the presence of intracellular neurofibrillary tangles made up of abnormally phosphorylated tau, a microtubule-associated protein, currently, most evidence suggests that Aβ plays the central role in the pathogenesis of the disease. Nevertheless, the etiology of AD is still poorly understood.

Recent advances in molecular genetics has suggested several genetic links to AD including mutations in the amyloid precursor protein (APP), the presenilin 1 protein, α-2 macroglobulin (A2M), nicastrin, and APOEε4. The chromosomal “hotspot” for late onset Alzheimer's disease (>65 years of onset, LOAD) has been mapped to 10 q. In contrast, the genetic loci for familial early onset Alzheimer's disease (<65 years of onset, EOAD) maps specifically to APP mutations at the γ-secretase site or mutations in the presenilin 1 gene known to affect γ-secretase activity. It is important to distinguish the difference between the genes linked to LOAD and EOAD. Most, if not all of the EOAD mutations found in presenilin, nicastrin, or the APP γ-cleavage site, are linked to y-secretase cleavage. On the other hand, the genes linked to LOAD have no common link to AD except for their ability to alter Aβ secretion from cells or clearance in the brain. Therefore, it seems clear that EOAD is caused by a specific defect in the γ-secretase activity, while the specific defect(s) in LOAD is still not clear.

The Aβ peptide is generated by the endoproteolytic cleavage of the amyloid precursor protein (APP), a large type I transmembrane protein. The two enzymes that cleave APP in the amylogenic pathway are called the β- and γ-secretases which cleave APP from the N- and C-termini, respectively. In this pathway, the β-secretase (BACE) is the rate limiting enzyme in the cleavage of APP, producing a sAPP-β fragment that is secreted from the cell and a C99 fragment that is left in the membrane. The C99 fragment is the substrate for the γ-secretase (GACE) which cleaves C99 to produce Aβ and a C99 “stub” that seems to function in a complex with Tip60 and Fe65 which derepresses a gene in the NFκ-B pathway through IL-1β, KAI1 (a tetraspanin cell surface molecule). The genetic, biochemical, and molecular evidence for AD suggests LOAD is likely to be polygenic and involve one or more genetic defects, familial and/or spontaneous.

APP processing involves different secretase enzymes: BACE cleavage produces sAPPβ and the C99 (or C89) fragment. The sAPPβ fragment is secreted out of the cells and C99 is the substrate for the γ-secretase. The γ-secretase then cleaves C99 into the amyloidgenic peptides Aβ40 or Aβ42. The α-secretase cleavage produces sAPPα and C83. The sAPPα is secreted out of the cell and the C83 fragment is cleaved by the γ-secretase into the nonamyloidgenic P3 peptide.

We have now surprisingly discovered heretofore unidentified modifiers of Aβ secretion; overexpression of these genes increased Aβ40 and A⊖42 expression and data indicate that they directly affect γ-secretase cleavage to increase the production of Aβ. Thus, these genes are potentially druggable therapeutic targets for pathological conditions associated with Aβ secretion, including but not limited to, Alzheimer's Disease (AD).

### SUMMARY OF THE INVENTION

The instant application relates to the discovery of several heretofore unknown modifiers of Aβ secretion and the use of said modifiers as targets for the development of new therapeutics to treat, prevent or ameliorate pathological conditions associated with Aβ secretion. Thus, in one aspect the invention relates to a method to identify modulators useful to treat, prevent or ameliorate said conditions, comprising: a) assaying for the ability of a candidate modulator, in vitro or in vivo, to inhibit the activity of a protein selected from the group consisting of those disclosed in Table 1 and/or inhibit the expression of a gene encoding a protein selected from the group consisting of those disclosed in Table 1 and which can further include b) assaying for the ability of an identified inhibitory modulator to reverse the pathological effects observed in animal models of said conditions and/ or in clinical studies with subjects with any one or more of said conditions.

In another aspect, the invention relates to a method to treat, prevent or ameliorate pathological conditions associated with Aβ secretion, comprising administering to a subject in need thereof an effective amount of one or more modulators of any one or more proteins selected from the group consisting of those disclosed in Table 1 wherein said modulator, e.g., inhibits the activity of said protein or inhibits the expression of a gene of said protein in said subject. In a further embodiment, the modulator comprises any one or more substances selected from the group consisting of antisense oligonucleotides, triple helix DNA, ribozymes, RNA aptamers, siRNA and double or single stranded RNA wherein said substances are designed to inhibit gene expression of any one or more proteins selected from the group consisting of those disclosed in Table 1. In a further embodiment, the modulator comprises antibodies to any one or more proteins selected from the group consisting of those disclosed in Table 1 or fragments thereof, wherein said antibodies can e.g., inhibit enzymatic or other protein activity. It is contemplated herein that one or more modulators of one or more of said proteins may be administered.

In another aspect, the invention relates to a method to treat, prevent or ameliorate pathological conditions associated with Aβ secretion, comprising administering to a subject in need thereof a pharmaceutical composition comprising an effective amount of one or more modulators of any one or more proteins selected from the group consisting of those disclosed in Table 1 wherein said modulator, e.g., inhibits the activity of said protein or inhibits the expression of a gene of said protein in said subject. In a further embodiment, the modulator comprises any one or more substances selected from the group consisting of antisense oligonucleotides, triple helix DNA, ribozymes, RNA aptamers, siRNA and double or single stranded RNA wherein said substances are designed to inhibit gene expression of any one or more proteins selected from the group consisting of those disclosed in Table 1. In a further embodiment, the modulator comprises antibodies to any one or more proteins selected from the group consisting of those disclosed in Table 1 or fragments thereof, wherein said antibodies can e.g., inhibit enzymatic or other protein activity. It is contemplated herein that one or more modulators of one or more of said proteins may be administered.

In another aspect, the invention relates to a pharmaceutical composition comprising one or more modulators of any one or more proteins selected from the group consisting of those disclosed in Table 1 in an amount effective to treat, prevent or ameliorate pathological conditions associated with Aβ secretion in a subject in need thereof wherein said modulator, e.g., can inhibit the activity of any one or more of said proteins and/or inhibit the gene expression of any one or more of said proteins. In a further embodiment, the modulator comprises any one or more substances selected from the group consisting of antisense oligonucleotides, triple helix DNA, ribozymes, RNA aptamers, siRNA and double or single stranded RNA wherein said substances are designed to inhibit gene expression of any one or more proteins selected from the group consisting of those disclosed in Table 1. In a further embodiment, the modulator comprises antibodies to any one or more proteins selected from the group consisting of those disclosed in Table 1 or fragments thereof, wherein said antibodies can e.g., inhibit enzymatic or other protein activity.

In another aspect, the invention relates to a method to diagnose subjects suffering from pathological conditions associated with Aβ secretion who may be suitable candidates for treatment with one or more modulators of any one or more proteins selected from the group consisting of those disclosed in Table 1 comprising detecting levels of said proteins in a biological sample from said subject wherein subjects with increased levels compared to controls would be a suitable candidate for modulator treatment.

In yet another aspect, the invention relates to a method to diagnose a subject suffering from pathological conditions associated with Aβ secretion who may be a suitable candidate for treatment with one or more modulators of any one or more proteins selected from the group consisting of those disclosed in Table 1 comprising assaying mRNA levels of said protein in a biological sample from said subject wherein a subject with increased mRNA levels compared to controls would be a suitable candidate for modulator treatment.

In yet another aspect, there is provided a method to treat, prevent or ameliorate pathological conditions associated with Aβ secretion comprising: (a) assaying a subject for mRNA and/or protein levels of a protein selected from the group consisting of those disclosed in Table 1; and (b) administering to a subject with increased levels of mRNA and/or protein levels compared to controls a modulator of any one or more of said proteins in an amount sufficient to treat, prevent or ameliorate said conditions.

In yet another aspect of the present invention, there are provided assay methods and kits comprising the components necessary to detect expression of polynucleotides encoding a protein selected from the group consisting of those disclosed in Table 1 or levels of any one or more of said proteins or fragments thereof, in body tissue samples derived from a patient, such kits comprising, e.g., antibodies that bind to any one or more of said proteins, or to fragments thereof, or oligonucleotide probes that hybridize with said polynucleotides. In a preferred embodiment, such kits also comprise instructions detailing the procedures by which the kit components are to be used.

The present invention also pertains to the use of a modulator for any one or more proteins selected from the group consisting of those disclosed in Table 1 in the manufacture of a medicament for the treatment, prevention or amelioration of pathological conditions associated with Aβ secretion. In one embodiment, said modulator comprises any one or more substances selected from the group consisting of antisense oligonucleotides, triple helix DNA, ribozymes, RNA aptamer, siRNA and double or single stranded RNA wherein said substances are designed to inhibit gene expression of any one or more of said proteins. In yet a further embodiment, said modulator comprises one or more antibodies to any one or more of said proteins, or fragments thereof, wherein said antibodies or fragments thereof can, e.g., inhibit enzymatic or other activity of said proteins.

The invention also pertains to a modulator of any one or more proteins selected from the group consisting of those disclosed in Table 1 for use as a pharmaceutical. In one embodiment, said modulator comprises any one or more substances selected from the group consisting of antisense oligonucleotides, triple helix DNA, ribozymes, RNA aptamer, siRNA and double or single stranded RNA wherein said substances are designed to inhibit the gene expression of any one or more of said proteins. In yet a further embodiment, said modulator comprises one or more antibodies to any one or more of said proteins, or fragments thereof, wherein said antibodies or fragments thereof can, e.g., inhibit enzymatic or other protein activity.

### DETAILED DESCRIPTION OF THE INVENTION

In practicing the present invention, many conventional techniques in molecular biology are used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

As used herein and in the appended claims, the singular forms “a”, “an”, and “the” include plural reference unless the context clearly dictates otherwise. Thus, for example, reference to the “antibody” is a reference to one or more antibodies and equivalents thereof known to those skilled in the art, and so forth.

“Pathological conditions associated with Aβ secretion” as used herein include, but are not limited to, conditions associated with abnormalities in the APP pathway, including but not limited to, modified APP metabolism or processing of components involved in the APP pathway, for example, abnormal α-, β-, or γ-secretase activity, and/or Aβ secretion which may be characterized by the formation of insoluble amyloid deposits (senile plaques), the major component of which is the 40-42 amino acid amyloid beta (Aβ) peptide, a proteolytic product of the amyloid precursor protein (APP). Such conditions include Alzheimer's Disease as well as other conditions characterized by degeneration and eventual death of neurons in brain clusters controlling memory, cognition and behavior. Such conditions may also include, but are not limited to, Parkinson's Disease, tauopathies, prion diseases, frontotemporal dementia, striatonigral degeneration, Lewd body dementia, Huntington's disease, Pick's disease, amyloidosis, and other neurodegenerative disorders associated with excess Aβ production.

“Nucleic acid sequence”, as used herein, refers to an oligonucleotide, nucleotide or polynucleotide, and fragments or portions thereof, and to DNA or RNA of genomic or synthetic origin that may be single or double stranded, and represent the sense or antisense strand.

The term “antisense” as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand combines natural sequences produced by the cell to form duplexes. These duplexes then block either the further transcription or translation. The designation “negative” is sometimes used in reference to the antisense strand, and “positive” is sometimes used in reference to the sense strand.

“cDNA” refers to DNA that is complementary to a portion of messenger RNA (mRNA) sequence and is generally synthesized from an mRNA preparation using reverse transcriptase.

As contemplated herein, antisense oligonucleotides, triple helix DNA, RNA aptamers, ribozymes, siRNA and double or single stranded RNA are directed to a nucleic acid sequence such that the nucleotide sequence chosen will produce gene-specific inhibition of gene expression. For example, knowledge of a nucleotide sequence may be used to design an antisense molecule which gives strongest hybridization to the mRNA. Similarly, ribozymes can be synthesized to recognize specific nucleotide sequences of a gene and cleave it (Cech. J. Amer. Med Assn. 260:3030 (1988)). Techniques for the design of such molecules for use in targeted inhibition of gene expression is well known to one of skill in the art.

The individual proteins/polypeptides referred to herein include any and all forms of these proteins including, but not limited to, partial forms, isoforms, variants, precursor forms, the full length protein, fusion proteins containing the sequence or fragments of any of the above, from human or any other species. Protein homologs or orthologs which would be apparent to one of skill in the art are included in this definition. It is also contemplated that the term refers to proteins isolated from naturally occurring sources of any species such as genomic DNA libraries as well as genetically engineered host cells comprising expression systems, or produced by chemical synthesis using, for instance, automated peptide synthesizers or a combination of such methods. Means for isolating and preparing such polypeptides are well understood in the art.

The term “sample” as used herein, is used in its broadest sense. A biological sample from a subject may comprise blood, urine, brain tissue, primary cell lines, immortilized cell lines, or other biological material with which protein activity or gene expression may be assayed. A biological sample may include, for example, blood, tumors or other specimens from which total RNA may be purified for gene expression profiling using, for example, conventional glass chip microarray technologies such as Affymetrix chips, RT-PCR or other conventional methods.

As used herein, the term “antibody” refers to intact molecules as well as fragments thereof, such as Fa, F(ab′)₂, and Fv, which are capable of binding the epitopic determinant. Antibodies that bind specific polypeptides can be prepared using intact polypeptides or fragments containing small peptides of interest as the immunizing antigen. The polypeptides or peptides used to immunize an animal can be derived from the translation of RNA or synthesized chemically, and can be conjugated to a carrier protein. Commonly used carriers that are chemically coupled to peptides include bovine serum albumin and thyroglobulin. The coupled peptide is then used to immunize an animal (e.g., a mouse, goat, chicken, rat or a rabbit).

The term “humanized antibody” as used herein, refers to antibody molecules in which amino acids have been replaced in the non-antigen binding regions in order to more closely resemble a human antibody, while still retaining the original binding ability.

A “therapeutically effective amount” is the amount of drug sufficient to treat, prevent or ameliorate pathological conditions associated with Aβ secretion.

“Subject” refers to any human or nonhuman organism.

Thus, in one aspect the invention relates to a method to identify modulators useful to treat, prevent or ameliorate pathological conditions associated with Aβ secretion including, but not limited to Alzheimer's Disease comprising: a) assaying for the ability of a candidate modulator to inhibit the activity of any one or more proteins selected from the group consisting of those disclosed in Table 1 and/or to inhibit the expression in vitro or in vivo of a gene encoding any one or more of said proteins and which can further include b) assaying for the ability of an identified inhibitory modulator to reverse the pathological effects observed in animal models of said conditions and/ or in clinical studies with subjects with any one or more of said conditions.

Conventional screening assays (both in vitro and in vivo) may be used to identify modulators that inhibit protein activity and/or inhibit gene expression. Protein activity levels, e.g., enzymatic activity levels, can be assayed in a subject using a biological sample from the subject using conventional enzyme activity assays. Gene expression (e.g. mRNA levels) may also be determined using methods familiar to one of skill in the art, including, for example, conventional Northern analysis or commercially available microarrays. Additionally, the effect of test compound inhibition of protein levels can be detected with an ELISA antibody-based assay or fluorescent labelling reaction assay. These techniques are readily available for high throughput screening and are familiar to one skilled in the art.

Data gathered from these studies would be used to identify those modulators with therapeutic usefulness for the treatment of pathological conditions discussed herein. Inhibitory substances could be further assayed in conventional live animal models familiar to one of skill in the art and/or in clinical trials with humans according to conventional methods to assess the ability of said compound to treat, prevent or ameliorate any one or more of said conditions in vivo.

Candidate modulators for analysis according to the methods disclosed herein include chemical compounds known to inhibit the proteins identified as modifiers herein as well as compounds whose effects on these proteins at any level have yet to be characterized. Compounds known to possess inhibitory activity could be directly assayed in animal models or in clinical trials as discussed above.

In another aspect, the invention relates to a method to treat, prevent or ameliorate pathological conditions associated with Aβ secretion including, but not limited to Alzheimer's Disease, comprising administering to a subject in need thereof a pharmaceutical composition comprising an effective amount of any one or more modulators of a protein selected from the group consisting of those disclosed in Table 1. Such modulators include antibodies directed to said proteins or fragments thereof. In certain particularly preferred embodiments, the pharmaceutical composition comprises antibodies that are highly selective for human forms of said proteins or portions thereof. Antibodies to said proteins may cause the aggregation of the proteins in a subject and thus inhibit or reduce protein activity, e.g. enzymatic activity. Such antibodies may also inhibit or decrease protein activity, for example, by interacting directly with active sites or by blocking access of substrates to active sites. Antibodies may also be used to inhibit protein activity by preventing protein-protein interactions that may be involved in the regulation of the protein and necessary for, e.g., enzymatic activity. Antibodies with inhibitory activity such as described herein can be produced and identified according to standard assays familiar to one of skill in the art.

Antibodies to the modifiers disclosed herein may also be used diagnostically. For example, one could use these antibodies according to conventional methods to quantitate levels of an modifier in a subject; abnormal levels compared to a suitable control could be indicative of various clinical forms or severity of any one or more pathological conditions disclosed herein. Such information would also be useful to identify subsets of patients with any one or more of said conditions that may or may not respond to treatment with inhibitors to said modifiers. Similarly, it is contemplated herein that quantitating the message level of an modifier disclosed herein in a subject would be useful for diagnosis and determining appropriate therapy; subjects with increased mRNA levels of any one or more of these proteins compared to appropriate control individuals would be considered suitable candidates for treatment with modulators as disclosed herein.

Thus in another aspect, the present invention relates to a diagnostic kit which comprises:
(a) a polynucleotide of a protein selected from the group consisting of those disclosed in Table 1 or fragments thereof;
(b) a nucleotide sequence complementary to that of (a);
(c) an RNAi sequence complementary to that of (a);
(d) a polypeptide selected from the group consisting of those disclosed in Table 1 or a fragment thereof; or
(e) an antibody to a polypeptide selected from the group consisting of those disclosed in Table 1, or a fragment thereof
   It will be appreciated that in any such kit, (a), (b), (c), (d) or (e) may comprise a substantial component.

Similarly, it is contemplated herein that monitoring levels or activity and/ or detecting gene expression (mRNA levels) of any one or more of the modifiers disclosed herein in a subject may be used as part of a clinical testing procedure, for example, to determine the efficacy of a given treatment regimen. For example, patients to whom a test substance has been administered would be clinically evaluated and patients in whom modifier levels, activity and/or gene expression levels are higher than desired (i.e. levels greater than levels in control patients or in patients in whom any one or more of said conditions has been sufficiently alleviated by clinical intervention) could be identified. Based on these data, the clinician could then adjust the dosage, administration regimen or type of therapeutic substance prescribed.

Factors for consideration for optimizing a therapy for a patient include the particular condition being treated, the particular mammal being treated, the clinical condition of the individual patient, the site of delivery of the active compound, the particular type of the active compound, the method of administration, the scheduling of administration, and other factors known to medical practitioners. The therapeutically effective amount of an active compound to be administered will be governed by such considerations, and is the minimum amount necessary for the treatment, prevention or amelioration of a pathological condition associated with Aβ secretion or modified APP metabolism as discussed herein.

Suitable antibodies to the proteins disclosed herein may be obtained from a commercial source or produced according to conventional methods. For example, described herein are methods for the production of antibodies capable of specifically recognizing one or more differentially expressed gene epitopes. Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

For the production of antibodies to the polypeptides discussed herein, various host animals may be immunized by injection with the polypeptides, or a portion thereof. Such host animals may include, but are not limited to, rabbits, mice, goats, chicken, and rats. Various adjuvants may be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as target gene product, or an antigenic functional derivative thereof For the production of polyclonal antibodies, host animals such as those described above, may be immunized by injection with the polypeptides, or a portion thereof, supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD, IgY and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived from a murine mAb and a human immunoglobulin constant region.

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce differentially expressed gene-single chain antibodies. Single chain antibodies are formed by linking the heavy and light chain fragments of the Fv region via an amino acid bridge, resulting in a single chain polypeptide.

Most preferably, techniques useful for the production of “humanized antibodies” can be adapted to produce antibodies to the polypeptides, fragments, derivatives, and functional equivalents disclosed herein. Such techniques are disclosed in U.S. Pat. Nos. 5,932,448; 5,693,762; 5,693,761; 5,585,089; 5,530,101; 5,910,771; 5,569,825; 5,625,126; 5,633,425; 5,789,650; 5,545,580; 5,661,016; and 5,770,429, the disclosures of all of which are incorporated by reference herein in their entirety.

Antibody fragments that recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity.

Detection of the antibodies described herein may be achieved using standard ELISA, FACS analysis, and standard imaging techniques used in vitro or in vivo. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, (3-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I ³⁵S or ³H.

Particularly preferred, for ease of detection, is the sandwich assay, of which a number of variations exist, all of which are intended to be encompassed by the present invention. For example, in a typical forward assay, unlabeled antibody is immobilized on a solid substrate and the sample to be tested brought into contact with the bound molecule. After a suitable period of incubation, for a period of time sufficient to allow formation of an antibody-antigen binary complex, a second antibody, labeled with a reporter molecule capable of inducing a detectable signal, is added and incubated, allowing time sufficient for the formation of a ternary complex of antibody-antigen-labeled antibody. Any unreacted material is then washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an antibody which is specific for the modifier polypeptide or fragments thereof.

The most commonly used reporter molecules are either enzymes, fluorophore- or radionucleotide-containing molecules. In the case of an enzyme immunoassay, an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of polypeptide or polypeptide fragment of interest which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established assays and are particularly preferred for the present method. However, other reporter molecules, such as radioisotopes, chemiluminescent or bioluminescent molecules may also be employed. It will be readily apparent to those skilled in the art how to vary the procedure to suit the required use.

The pharmaceutical compositions of the present invention may also comprise substances that inhibit the expression of disclosed modifiers at the nucleic acid level. Such molecules include ribozymes, antisense oligonucleotides, triple helix DNA, RNA aptamers, siRNA and/or double or single stranded RNA directed to an appropriate nucleotide sequence of nucleic acid encoding a modifier. These inhibitory molecules may be created using conventional techniques by one of skill in the art without undue burden or experimentation. For example, modifications (e.g. inhibition) of gene expression can be obtained by designing antisense molecules, DNA or RNA, to the control regions of the genes encoding the polypeptides discussed herein, i.e. to promoters, enhancers, and introns. For example, oligonucleotides derived from the transcription initiation site, e.g., between positions −10 and +10 from the start site may be used. Notwithstanding, all regions of the gene may be used to design an antisense molecule in order to create those which gives strongest hybridization to the mRNA and such suitable antisense oligonucleotides may be produced and identified by standard assay procedures familiar to one of skill in the art.

Similarly, inhibition of the expression of gene expression may be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). These molecules may also be designed to block translation of mRNA by preventing the transcript from binding to ribosomes.

Ribozymes, enzymatic RNA molecules, may also be used to inhibit gene expression by catalyzing the specific cleavage of RNA. The mechanism of ribozyme action involves sequence-specific hybridization of the ribozyme molecule to complementary target RNA, followed by endonucleolytic cleavage. Examples which may be used include engineered “hammerhead” or “hairpin” motif ribozyme molecules that can be designed to specifically and efficiently catalyze endonucleolytic cleavage of gene sequences. Specific ribozyme cleavage sites within any potential RNA target are initially identified by scanning the target molecule for ribozyme cleavage sites which include the following sequences: GUA, GUU and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Ribozyme methods include exposing a cell to ribozymes or inducing expression in a cell of such small RNA ribozyme molecules (Grassi and Marini, 1996, Annals of Medicine 28: 499-510; Gibson, 1996, Cancer and Metastasis Reviews 15: 287-299). Intracellular expression of hammerhead and hairpin ribozymes targeted to mRNA corresponding to at least one of the genes discussed herein can be utilized to inhibit protein encoded by the gene.

Ribozymes can either be delivered directly to cells, in the form of RNA oligonucleotides incorporating ribozyme sequences, or introduced into the cell as an expression vector encoding the desired ribozymal RNA. Ribozymes can be routinely expressed in vivo in sufficient number to be catalytically effective in cleaving mRNA, and thereby modifying mRNA abundance in a cell (Cotten et al., 1989 EMBO J. 8:3861-3866). In particular, a ribozyme coding DNA sequence, designed according to conventional, well known rules and synthesized, for example, by standard phosphoramidite chemistry, can be ligated into a restriction enzyme site in the anticodon stem and loop of a gene encoding a tRNA, which can then be transformed into and expressed in a cell of interest by methods routine in the art. Preferably, an inducible promoter (e.g., a glucocorticoid or a tetracycline response element) is also introduced into this construct so that ribozyme expression can be selectively controlled. For saturating use, a highly and constituently active promoter can be used. tDNA genes (i.e., genes encoding tRNAs) are useful in this application because of their small size, high rate of transcription, and ubiquitous expression in different kinds of tissues.

Therefore, ribozymes can be routinely designed to cleave virtually any mRNA sequence, and a cell can be routinely transformed with DNA coding for such ribozyme sequences such that a controllable and catalytically effective amount of the ribozyme is expressed. Accordingly the abundance of virtually any RNA species in a cell can be modified or perturbed.

Ribozyme sequences can be modified in essentially the same manner as described for antisense nucleotides, e.g., the ribozyme sequence can comprise a modified base moiety.

RNA aptamers can also be introduced into or expressed in a cell to modify RNA abundance or activity. RNA aptamers are specific RNA ligands for proteins, such as for Tat and Rev RNA (Good et al., 1997, Gene Therapy 4: 45-54) that can specifically inhibit their translation.

Gene specific inhibition of gene expression may also be achieved using conventional double or single stranded RNA technologies. A description of such technology may be found in WO 99/32619 which is hereby incorporated by reference in its entirety. In addition, siRNA technology has also proven useful as a means to inhibit gene expression (Cullen, B R Nat. Immunol. 2002 Jul.;3(7):597-9; Martinez, J. et al. Cell 2002 Sep.6;110(5):563).

Antisense molecules, triple helix DNA, RNA aptamers, dsRNA, ssRNA, siRNA and ribozymes of the present invention may be prepared by any method known in the art for the synthesis of nucleic acid molecules. These methods include techniques for chemically synthesizing oligonucleotides such as solid phase phosphoramidite chemical synthesis. Alternatively, RNA molecules may be generated by in vitro and in vivo transcription of DNA sequences encoding the genes of the polypeptides discussed herein. Such DNA sequences may be incorporated into a wide variety of vectors with suitable RNA polymerase promoters such as T7 or SP6. Alternatively, cDNA constructs that synthesize antisense RNA constitutively or inducibly can be introduced into cell lines, cells, or tissues.

Vectors may be introduced into cells or tissues by many available means, and may be used in vivo, in vitro or ex vivo. For ex vivo therapy, vectors may be introduced into stem cells taken from the patient and clonally propagated for autologous transplant back into that same patient. Delivery by transfection and by liposome injections may be achieved using methods that are well known in the art.

In addition, the cDNA and/or protein of the modifiers identified herein can be used to identify other proteins, e.g. receptors, that are modified by these modifiers in tissues in vivo. Proteins thus identified can be used for drug screening to treat pathological conditions associated with Aβ secretion. To identify these genes, including those that are downstream of the modifiers, it is contemplated, for example, that one could use conventional methods to treat animals in conventional in vivo models of any one or more said pathological conditions with a specific inhibitor of an modifier, sacrifice the animals, remove tissue samples and isolate total RNA from the tissue and employ standard microarray assay technologies to identify message levels that are altered relative to a control animal (animal to whom no inhibitor has been administered).

Conventional in vitro or in vivo assays may be used to identify possible genes that lead to over expression of the modifiers identified herein. The genes thus identified can be used to screen drugs that might be potent therapeutics for the treatment of pathological conditions associated with Aβ secretion. For example, a conventional reporter gene assay could be used in which the promoter region of a modifier gene is placed upstream of a reporter gene, the construct transfected into a suitable cell (for example, a tumor cell line such as HeLa, CHO, or HEK293 or primary cells such as human diploid fibroblasts, endothelial or chondrocyte cells) and using conventional techniques, the cells assayed for an upstream gene that causes activation of the modifier promoter by detection of the expression of the reporter gene.

It is contemplated herein that one can inhibit the function and/or expression of a gene or protein modified by a modifier identified herein as a way to treat the pathological conditions discussed herein by designing, for example, antibodies to these proteins and/or designing inhibitory antisense oligonucleotides, triple helix DNA, ribozymes, ssRNA, dsRNA, siRNA and RNA aptamers targeted to the genes for such proteins according to conventional methods. Pharmaceutical compositions comprising such inhibitory substances for the treatment of the pathological conditions discussed herein are also contemplated.

The pharmaceutical compositions disclosed herein useful for treating, preventing and/or ameliorating pathological conditions associated with Aβ secretion are to be administered to a patient at therapeutically effective doses. A therapeutically effective dose refers to that amount of the compound sufficient to result in the treatment, prevention, or amelioration of any one or more of said conditions and would be able to be determined by a clinician or other person possessing ordinary skill in the art.

The inhibitory substances of the present invention can be administered as pharmaceutical compositions. Such pharmaceutical compositions for use in accordance with the present invention may be formulated in a conventional manner using one or more physiologically acceptable carriers or excipients.

Thus, compounds and their physiologically acceptable salts and solvates may be formulated for administration by inhalation or insufflation (either through the mouth or the nose) or topical, oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

Compounds may also be formulated in rectal compositions such as suppositories or retention enemas, e.g., containing conventional suppository bases such as cocoa butter or other glycerides.

Compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. A dose may be formulated in animal models to achieve a circulating plasma concentration range that includes the IC₅₀ (i.e., the concentration of the test compound that achieves a half-maximal inhibition of symptoms). Such information can then be used to determine useful doses and routes for administration in humans.

A therapeutically effective dose refers to that amount of active ingredient, for example, inhibitory compound, antisense oligonucleotides, triple helix DNA, ribozymes, RNA aptamer, siRNA or double or single stranded RNA designed to inhibit the expression of a gene encoding an modifier, antibodies to said modifiers or or fragments thereof, useful to treat, prevent and/or ameliorate pathological conditions associated with Aβ secretion. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

It is contemplated that the invention described herein is not limited to the particular methodology, protocols, and reagents described as these may vary. It is also to be understood that the terminology used herein is for the purpose of describing particular embodiments only, and is not intended to limit the scope of the present invention in any way.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices and materials are now described. All publications mentioned herein are incorporated by reference for the purpose of describing and disclosing the materials and methodologies that are reported in the publication which might be used in connection with the invention. Persons skilled in the art will recognize, or be able to ascertain using no more than routine experimentation, many equivalents to the specific embodiments of the invention described herein. Such equivalents are intended to be encompassed by the claims.

### EXAMPLES

The following methods are performed to conduct the examples disclosed below:

Construction of a 479 Gene Rearray

Approximately 20,000 clones in a proprietary full length cDNA clone collection are analyzed in silico based on their chromosomal location and putative function; 479 genes from the collection are found to map to the chromosome 10 q late onset Alzheimer's Disease “hotspot” with some level of annotation. To further annotate these genes at a functional level, the 479 protein sequences are run in three distinct sequence analysis programs with alternative search algorithms: a proprietary program referred to as InterProScan, Celera/Panther protein family classification databases and Basic local alignment search tool (BLAST) where the 479 clones are screened against the non-redundant (nr) protein database at NCBI (Altschul, S. F. et al., (1990) J. Mol. Biol. 215:403-410). The data from all three methods is overlapped and a complete annotation of each gene compiled in a database.

The DNA for the 479 10 q genes are rearrayed from bacterial E. coli strain DH5α (Invitrogen, Carlsbad, Calif.) glycerol stocks from an approximately 20,728 proprietary gene set. The set is contained in 384-well Genetix plates (Genetix USA Inc., St. James, N.Y.) containing 60 μl of bacterial glycerol stock (Luria broth (LB) in 8% glycerol). A 2 μl aliquot of each well of interest is used to inoculate a single Greiner 384-deep well plate (Greiner BioONE Inc., Longwood, Fla.) containing 100 μl of LB/8% glycerol. The plate is sealed with an airpore sheet (Qiagen, Valencia, Calif.), wrapped with Saran, and incubated at 37° C. for 22 hours without shaking. A 5 μl aliquot of this culture is inoculated into 1 ml of terrific broth (TB) containing 100 μg/μl ampicillin (arrayed in five Qiagen deep 96-well plates covered with airpore sheets) and grown for 22 hours at 37° C. shaking at 250 rpm. To improve the density of the cultures, 100 μl of the inoculate is reinoculated into another Qiagen deep 96 well plate containing fresh 1×TB 100 μg/μl ampicillin and grown overnight. The cells are spun down at 4000 rpm for 15 minutes and the supernatants are removed. The cell pellets are transferred onto a Qiagen BioRobot 8000 where DNA minipreps are prepared using the QIAprep Turbo96 PB (1-4 plate) protocol. The protocol uses Corning 96 well UV-plates (Corning Inc. Life Sciences, Acton, Mass.) in place of Qiagen 96-well plates for the DNA elution. The DNA concentrations are determined according to conventional methods using A260/280 ratios calculated on a SPECTRAmax 190 (Molecular Devices Corporation, Sunnyvale, Calif.).

To determine the volume of DNA needed to transfect plates in a 96 well format, the average DNA concentration of each plate is calculated. The average DNA concentration of the “mother plate” is used to resuspend each well in a volume of 180 μl to come up with an average DNA value of 25 ng/μl. A 6 μl volume from each well is plated into 30 fresh 96 well PCR plates (Coming Inc. Life Sciences, Acton, Mass.). Each of these transfection ready “daughter plates”, a copy of the “mother plates,” is heat sealed and frozen at −20° C.

Construction of a 2268 Gene Rearray

A 2268 gene rearray is generated to enrich the 20,720 genes into a small subset of genes that seemed to be the best potential drug targets based on annotation. This function based rearray is created by identifying gene annotation that matched the key words “kinase”, “phosphatase”, “protease”, “apoptosis”, “eicosanoid metabolism”, “sphingolipid metabolism”, “polyamine metabolism”, “phospholipase”, or “chemokines.” From this search all redundant clones are removed leaving the resulting set of genes in the rearray.

Another set of genes in the rearray is called CGUFs, or conserved genes (to C. elegans, and D. Melanogaster) of unknown function. This list of genes is derived from the Celera database of 613 predicted human genes with orthology to fly and worms with no assigned function. The definition of orthology is when two sequences are each other's best BLAST hit (a P value of 1 e⁻¹⁰). The 613 Celera genes are then Blasted against the clone collection using Blastn (Altschul, S F et al. Nucleic Acids Res. 1997 25:3389-3402). The clones are compared to LifeSeqGold (Incyte Pharmaceuticals, Palo Alto, Calif.), which includes assembled EST sequences that may represent experimentally derived transcripts and splice variants. 529 of the original 613 are found in our proprietary clone collection and two to three genes for each of the 529 genes are represented in the 2268 rearray to increase the chance of obtaining a full coding region. The kinases are filtered further by a keyword search by blasting against the REFSeq database (NCBI, Bethesda, Md.) with a requirement that the 5′ end of the coding region matches the REFSeq entry with 95% identity over 50 base pairs. The E2s are filtered using a tBlastn of the clones against the GenBank database. Hits that scored ≦1 e⁻¹⁰ are added to the rearray. This rearray is prepared in the same fashion as the 479 rearray except that six, rather than one 384 well plate is used to grow up the bacteria to prepare the DNA minipreps.

Transfections

CHO K1 cells (ATCC, Manassas, Va.) are plated with DMEM, 10% Fetal Bovine Serum, 5% Penn/Strep, and 22 mg of L-Proline (Sigma Chemical, St. Louis, M0.) into sterile, covered, 96 well dishes (Corning Inc. Life Sciences, Acton, Mass.) at 10,000-cells/75 μl/well using a Multidrop dispenser according to the manufacturer's protocol (Thermo Labsystems, Franklin, Mass.). The plates are incubated overnight at 37° C. in water jacked CO₂ cell culture chambers. For the 479 gene array, transfection mixtures are prepared with ˜200 ng of cDNA, 10 μl of OptiMEM, and 1 μl of FUGENE 6 per well of a 96 well plate (Roche Applied Sciences, Indianapolis, Ind.). The cDNA of interest is co-transfected with full length APP in a 1:15 ratio (cDNA:APPwt(695)) according to conventional methods.

For the HEK 293 cells (ATCC, Manassas, Va.) transfections, Qiagen® SuperFect reagent is used according to the manufacturer's directions. In 6-well dishes, 5×10⁵ cells are plated with DMEM, 10% Fetal Bovine Serum, 5% Penn/Strep (Sigma Chemical, St. Louis, Mo.) and grown up for 24 hours. The SuperFect mix is made up with 100 μl of serum free medium (DMEM), 3 μg of total DNA, and 20 μl of SuperFect. The media is removed from the cells and 1 mL of fresh media is added. The entire SuperFect mix is added to the media and incubated at 37° C. for 2 hours. The mixture is then removed and the cells are washed once with 3 mL of PBS. Fresh media is added back to the cells and they are incubated for 24 or 48 hours.

For the 2268 gene array, transfection efficiency is normalized against the pGL3-control vector (Promega Madison, Wis. cat#E1741) included in the transfection mixture to a final concentration of 27 ng/well. A total of 25 plates or 2400 wells is needed for the transfection of 2268 genes. To prepare the master mix for this reaction, 35 mls of OptiMEM, 375 μl of APPwt DNA (1 μg/μl), 94 μl of pGL3-control vector (1 μg/μl) are mixed in a 50 ml Falcon tube (Fisher Scientific, St. Louis, Mo.). 8 mls are taken from this mixture and added to 800 μl of FUGENE 6 and 75 μl of the new mix is plated in 4 96-well PCR plates. This plate is used on the BioMekFX (Coulter, Fullerton, Calif.) where 10 μl/well is mixed with the transfection ready DNA “daughter plates” with an average DNA concentration of 150 ng/well (6 μl of the “mother plate”). The FUGENE and media mixture added to the cDNA samples are mixed by pipetting 7 times. The mixtures are incubated for 20 minutes at room temperature and the complete volume added to the plated cells and mixed well. The cells and transfection mixture are incubated in the plates at 37° C. for 24 hours. The 24 hour time point was previously determined by conventional Western blot analysis to be the point where BACE overexpression and Aβ secretion from CHO K1 cells is maximal (data not shown).

Luciferase Assay

After 24 hours, the cell supernatants are removed from the plates and transferred into the conventionally precoated ELISA plates. At the same time, 100 μl of fresh complete DMEM (Sigma Chemical, St. Louis, Mo.) is added back onto the cells. Using the standard protocol from the BRIGHTGLO luciferase assay system (Promega Madison, Wis.), 90 μl of fresh luciferase reagent is added to each well and mixed. After a 2-minute room temperature incubation, each plate is read on a LUMINOSKAN ASCENT (Thermo Labsystems, Fullerton, Calif.) luminometer using a 500 ms integration time. The normalization procedure involves dividing the ELISA read out by the luciferase read out to determine fold induction of the assay. In this way, every transfection event is normalized to transfection efficiency. The normalization procedure also serves as a benchmark for cell viability.

Hit Validation

The clones that scored as “hits” (all genes that increased Aβ secretion ≧5 fold in the normalized data set and >1.5 fold in the raw data set) in the assay are retrieved from the “mother plate” glycerol stocks. Each stock is streaked out on a 1× LB agar/100 μg/ml ampicillin plate and grown overnight at 37° C. Three independent colonies are picked and grown up as 5 ml cultures in 15 ml Falcon tubes overnight at 37° C. DNA is prepared from these cultures using the HighSpeed Maxi Kit (Qiagen cat#12663) according to the manufacturer's protocol and the DNA concentration is determined by conventional methods on a Spectramax 190 (Molecular Devices). Every “hit” is then sequenced (Solvias, Basel, Switzerland) to confirm the 5′ and 3′ sequence of the gene. If the sequence corresponds to the correct gene, these DNAs are used to repeat the entire assay for a follow up validation. In these experiments, both Aβ40 and Aβ42 peptides are assayed using the ELISA and total Aβ levels are examined by Western blot analysis according to conventional methods.

SEAP Assay

One possible caveat of measuring Aβ peptides in cell culture media is the possibility that the overexpression of a gene could cause a general increase in secretion resulting in more Aβ release. To control for these nonspecific affects on secretion, a SEAP (Great EscAPe SEAP kit, Clontech Cat#PT3057-2) assay is used to measure total secretion of the transfected cells. Following the manufacturer's instructions, the cells are transfected with each cDNA of interest along with SEAP (100 ng/well). After a 48-hour incubation, 100 μl of assay buffer is added to each well followed by 100 μl of the chemiluminescent substrate and the samples incubated for 10 minutes. A control curve of alkaline phosphatase enzyme is measured over a 11-fold dilution to determine the linear range of detection of the assay. The plate is read on the LUMINOSKAN ASCENT (Thermo Labsystems) luminometer using a 500 ms integration time.

ELISA Antibodies

A commercially available mouse monoclonal antibody directed to the NH₂ terminus of the Aβ peptide is used as the capture antibody in pre-coated 96 well plates (Biosource Cat#KBH3481/PPO81 for Aβ40 and Cat#KBH3441/PPO81 for Aβ42). Polyclonal detection antibodies are obtained from Biosource (anti-hAβ40 Cat#44-348 and anti-hAβ42 Cat#44-344) and diluted 1/220 in 15 mM sodium azide. The secondary antibody (Biosource Cat#KBH3481 for Aβ40 and Cat#KBH3441 for Aβ42) is a horseradish peroxidase labeled anti-rabbit IgG. The secondary antibody is diluted 1/100 in 3.3 mM thymol.

Indirect Two Sandwich ELISA

The antibody-coated plates are washed 4× in PBS-TE (1 mM EDTA and 0.05% Tween 20, wash buffer) on a microplate washer (Bioteck Instruments, Inc, Winooski, Vt.) prior to use. 100 μl of the transfected cell's conditioned media is removed and diluted 1:2 in sample diluent containing 1 mM AEBSF (Biosource, Camarillo, Calif.). 100 μl of this mixture is added to the washed, antibody coated 96 well plate, covered with tape, and incubated at 4° C. overnight. The samples are removed and the plates are washed 4× with wash buffer. Detection antibody solution is added at 100 μl/well and the plates are incubated at room temperature for 2 hours while shaking. The plates are washed again 4× with wash buffer and the secondary antibody solution is added at 100 μl/well and incubated for 2 hours while shaking. The plates are washed 5× in wash buffer and patted dry on a paper towel. 100 μl of stabilized chromogen (tetramethylbenzidine) is added to each well and the plate is incubated for 30 minutes in the dark. 100 μl of stop solution (1N H₂S) is added to the plates to stop the reaction. The plates are read on a microplate reader at 450 nM (Molecular Devices) within one hour.

Robot Protocols

All transfections and ELISAs are run on a Beckman Coulter Biomek FX robot. The protocols are written in Visual Basic (Microsoft, Redmond, Wash.) and run on Biomek FX 2.1a software. The basic transfection procedure consists of two pipetting steps. First, 15 μl of the 96 well rearrayed cDNA plates are transferred into fresh 96 well plates containing 11 μl of serum free media (DMEM), full length APP, and FUGENE 6. Second, after the FUGENE mixture is incubated 20 minutes at room temperature, the entire mixture (25 μl) is transferred to the 75 μl cell solution. The plates are then incubated 24 hours at 37° C.

Northern Blot Analysis

Cyclophilin F cDNA (commercially available) is double digested with EcoRI and Not I for 3 hours at 37° C. The digest is resolved on a 1×TAE gel (BioRad Cat#161-3044) and the fragment excised with a razor according to conventional methods. The cDNA is extracted from the gel fragment using a spin column (Sigma Cat#S-6501, St. Louis, Mo.). The cDNA is labeled with fresh (<2 weeks old) P³² (Amersham Cat# REDIV/03, Piscataway, N.J.) using the REDIPRIME II kit (Amersham Cat #RPN1634) following the manufacturer's instructions.

A brain specific normalized Multi-Tissue Northern blot (MTN, Clontech Cat#7755-1, Palo Alto, Calif.) containing eight distinct regions of the brain (cerebellum, cerebral cortex, medulla, spinal cord, occipital pole, frontal lobe, temporal lobe, and putamen) is probed with the P³² labeled cyclophilin F cDNA. All incubation and wash steps are followed as described in the MTN user manual. The blot is exposed to X-ray film (Amersham Cat# RPN 3114k) at −70° C. for 24, 48, & 72 hours prior to development. The blot is stripped according to the manufacture's instructions and stored at −20° C. in saran wrap.

Additionally, a Human Multiple Tissue Expression Array (MTE, Clontech Cat#7776-1 Palo Alto, Calif.) is also probed with a P³² labeled cyclophilin F cDNA. This array contains 73 tissues including 20 distinct regions of the brain. The blot is probed, washed, and stripped according to the manufacturer's instructions.

Plasmids

The cDNA for APP wild type and the APP Swedish mutant are inserted into the pRK plasmid expression vector downstream of a cytomegalovirus promoter as previously described (Promega, Madison, Wis.) (Bodendorf, U., Fischer, F., Bodian, D., Multhaup, G., Paganetti, P. 2001 J. Biol. Chem. 276:12019 12023).

The full-length BACE cDNA was previously isolated from a human brain library and cloned into the pRK expression vector (Fischer, F., Paganetti, P. Brain Res. 1996. 716:91-100.) A C99 overexpression construct was created according to conventional methods (Invitrogen PAN neuronal library).

Western Blot Antibodies

To validate the ELISA phenotype of the “hits,” 10 μl of supernatant from each hit is run on an 18% Tris-HCl Criterion Gel (BioRad, Hercules, Calif.), transferred to a PVDF membrane (Millipore Immobilon P cat#1PVH0010, Bedford, Mass.) using a semi-dry apparatus (BioRad), the membrane is probed with a 1/2000 dilution of the 6E10 antibody (Signet, Dedham, Mass.) and a 1/2000 dilution of anti-mouse IgG conjugated to HRP (Pierce, Rockford, Ill.). Detection of full-length APP is used as the loading control.

Using conventional methods, the rabbit polyclonal antiserum 818 is raised against a synthetic antigen corresponding to peptides 484-501 of the β-secretase, BACE501. Antiserum 818 is affinity purified using commercially available reagents with the corresponding covalently coupled peptide (Bodendorf, U., Fischer, F., Bodian, D., Multhaup, G., Paganetti, P. 2001 J. Biol. Chem. 276:12019 12023). All antisera reacted equally well against BACE501. The monoclonal antibody β1 is raised as described previously (Fischer, F., Paganetti, P. Brain Res. 1996. 716:91-100.)) and reacts with the N-terminus of Aβ (residues 1-16). The mouse monoclonal antibody 6E10 is obtained from Signet, Dedham, Mass. and reacts with epitopes located between residues 1 and 16 in the amino terminus of Aβ. APPC8 antibody is raised against the C-terminal end of APP (amino acids 676-695) and it recognizes APP and all C-terminal fragments thereof (Fischer, F., Paganetti, P. Brain Res. 1996. 716:91-100.). The neopeptide rabbit antisera specific to the carboxy-terminus of sAPPβ (antisera 879) wild-type is raised against the synthetic peptide (Cys-Ile-Ser-Glu-Val-Lys-Met) (Bodendord, U., et al. 2002. J Neurochem. 80(5):799-806).

Cell Extraction Procedure and Western Blot Analysis

Cultured HEK 293 cells are extracted at 24 or 48 hr post-transfection in RIPA buffer (10 mM Tris, pH 7.5, 150 mM sodium chloride, 1 mM EDTA, 1% Nonidet P-40, 0.5% sodium-deoxycholate, 1% SDS) containing protease inhibitors (Complete® Roche Molecular Biochemicals, Indianapolis, Ind.) and centrifuged at 4° C. for 10 min at 10,000×g. The supernatants are collected and the pellets discarded. Subsequently, the cell extracts are resolved by SDS polyacrylamide gel electrophoresis, transferred to PVDF Immobilon-P® membranes (Millipore, Bedford, Mass.), and probed with primary antibodies. Supernatants are separated on 16% Tricine gels (Invitrogen Novex, Carlsbad, Calif.) to examine the metabolites sAPPβ & sAPPα. Using conventional methods, cell extracts are also separated on 16% Tricine gels (Invitrogen Novex, Carlsbad, Calif.) to examine the carboxy-terminal metabolites C99, C89, C83, APP, and BACE. Aβ is detected in the cell supernatants on 18% Tris-Tricine gels (BioRad Criterion, Hercules, Calif.) according to conventional methods. Membranes are blocked with 5% (w/v) low-fat milk powder in phosphate buffered saline, 0.05% Tween-20 (PBST), and incubated overnight at 4° C. with the primary antibody diluent. Bound antibodies are detected with goat anti-mouse or anti-rabbit IgG (Chemicon, Temecula, Calif.) conjugated to horseradish peroxidase diluted in PBST. Immunological detection is carried out with the ECL detection system (Amersham Pharmacia Biotech, Piscataway, N.J.) as previously described (Manni, M., et al., 1998. FEBS. 427:367-370).

### Example 1

### Chromosome 10 q Screen

The first step in our analysis was to define two rearrayed sets of clones that represent the best possible AD targets. From an initial proprietary collection of approximately 20,720 genes, a 2,268 rearray was generated enriched in CGUFs, kinases, phosphatases, proteases, and apoptosis related genes. The CGUF's are important in identifying genes that have homology to model organisms where detailed biochemical and molecular analysis of phenotypes can be carried out. The kinases, phosphatases, proteases, apoptosis related genes, and others were picked because they are putative drug targets. Based on this rationale, it is likely that genes from this rearray that modify Aβ secretion could be directly screened against compound libraries.

In contrast to the functional/druggable rearray, we also wanted to isolate all possible clones known to map to the LOAD locus found on chromosome 10 q (Taner, N. E., et al. 2000. Science. 290:2303-2304. Bertram, L., et al., Science. 2000. 290:2302-2303. Myers, A. et al., Science. 2000. 290:23042305.). First, STS markers adjacent to the markers used in the linkage analysis of chromosome 10 were mapped to the Celera genome sequence by Blastn sequence. This map was defined to include only the long arm of chromosome 10, the “hot spot”. Both the DNA and protein sequence set from this region was compared to our proprietary clone collection using conventional tBlastn and Blastp searches, respectively. All the clones that fit this criteria were compiled in a database and rearrayed into a small subset of 479 clones.

To further characterize the 479 clones in this region, a functional annotation analysis of these genes was performed using a “overlap cluster” approach by taking three different sequence analysis programs that access distinct databases and overlapping the information from each program. In this way, the most complete and accurate annotation could be achieved quickly. We first implemented the InterProScan program to examine homology of the proteins to domains in the InterPro databases (HMMPfam, HMMTigr, HMMSmart, BlastProDom, FingerPRINTScan, & ProfileScan). Next, we overlapped the InterPro results with the preannotated proteins from the proprietary Celera databases. Finally, we compared these two analyses with a Blastp search against the non-redundant public database. From this work, we were able to functionally annotate 625 genes or 56.8% of the 1101 predicted transcripts in the 10 q region. Only 479 full length clones of the 1101 possible transcripts are available to test. Of the 479, 354 are fully annotated (74%), while the rest are of unknown function (data not shown).

We screened the 479 gene set from chromosome 10 q first based on the linkage analysis to AD. Using FUGENE 6 (Roche Biochemicals, Indianapolis, Ind.) transfection reagent, log phase CHO K1 cells are transfected with ˜200 ng of each cDNA in 96 well plates. We had already determined that 24 hours was the optimal time point to measure Aβ levels by this ELISA (data not shown). Briefly, CHO K1 cells are transfected using the manufactures protocol, and Aβ levels are determined from the supernantant over a time course; 24, 48, and 72 hours. The linear range of detection was found to be 24 hours. Twenty four hours posttransfection, we harvested the supernatants from the cells and plated the media onto antibody coated ELISA plates. Transfection of the BACE enzyme is used as the positive control and vector alone is used as the negative control. The ELISA read out for Aβ40 and Aβ42 was repeated and indicated that Aβ40 and 42 levels are greater than controls in the BACE transfected cells by 4-fold and 2--fold respectively. We used the APPwt rather than the APPswe mutant because we wanted to observe the affects of these genes in the wild type state rather than a mutant state. We predicted that genes that increase Aβ with APPwt are more physiologically relevant for late onset AD than the APPswe which only represents a small subset of familial disease cases.

The Aβ40 screen resulted in a single gene causing an increase in Aβ40 levels (hit rate 0.0021%) while no genes affected Aβ42 secretion. This affect was repeated in subsequent ELISA assays indicating this affect was reproducible. This clone was sequenced and confirmed to be the cyclophilin F gene. Cyclophilin F is a member of the cyclophilin (class immunophilin) gene family of peptidyl-prolyl cis-trans isomerases (PPIases) that are known to bind the drug cyclosporin A (CsA)(Schreiber, S. L. (1991). Science. 251: 283. Bergsma, D. et al. (1991) J. Biol. Chem. 266: 23204-23214.). Besides binding to CsA, these proteins are thought to be involved in protein folding and/or intracellular protein transport. CsA is an immunosuppressive drug used to prevent graft vs. host disease during transplantation (Schreiber, S. L. and Crabtree, G. R. (1992). Immunol. Today. 13: 136.). CsA binds to its intracellular target, cyclophilin A (CPA) (and possibly other immunophilins), that inhibit effector molecules involved in intracellular signal transduction (Schreiber, S. L. and Crabtree, G. R. (1992). Immunol. Today. 13: 136.). The CsA/CpA complex can bind and inhibit the serine-threonine phosphatase calcineurin selectively blocking the transcription of early T-cell specific genes. This inhibition results in a block of T-cell activation and production of growth factors like IL-2.

To assess the potential function of cyclophilin F, we aligned the primary amino acid sequences of CpA, B, C, and F (data not shown). It is clear from alignment that cyclophilin F is a member of this immunophilin family. The residues that are known to bind CsA (based on the crystal structure) are 100% conserved in these proteins. This implicates CpF as a PPIase and CsA binding protein.

Cyclophilin F was found in our screen because of its physical location on chromosome 10 q. We mapped its exact location relative to the hot spot locus on 10 q. It maps just distal to the centromere and right of the highest peak between markers D10S1220 and D1051670 (data not shown).

### Example 3

### Cyclophilin F Validation

Western blot assay was used to confirm the ELISA cyclophilin F results. CHO K1 cells are transfected with a range of cyclophilin F concentrations in order to measure the gene dosage affect on Aβ levels. Since cyclophilin F is overexpressed from a CMV promoter (Invitrogen, Gateway®, Carlsbad, Calif.) one can assume the more DNA in the transfection the greater the expression in vivo. Our results indicate that a direct correlation exists between the gene dosage of cyclophilin F and the level of extracellular Aβ (data not shown). This is an interesting discovery because it suggests that the more cyclophilin F expressed, the more Aβ secreted.

A brain specific multi tissue Northern blot (MTN) blot (Clontech, Palo Alto, Calif.)) and a MTE blot (Clontech) containing 20 distinct regions of the brain was used to investigate cyclophilin F expression in this organ. Results of this analysis indicate that cyclophilin F is expressed in the brain, more specifically in the cerebellum, medulla, and spinal chord (predicted transcript of cyclophilin F is ˜0.624 kb) (data not shown). To a lesser extent, there is cyclophilin F expression in the cerebral cortex, temporal lobe, and the putamen (basal ganglia) shown in longer exposures. There also seems to be a longer mRNA species detected at 3.5 kb. This could be a possible splice variant or another cyclophilin message with homology to cyclophilin F.

The MTE blot was also probed to determine where cyclophilin F is expressed throughout the body as well as in regions of the brain not represented on the MTN blot. Data from a 72 hour exposure indicates that cyclophilin F is expressed in the temporal lobe, cerebellum, putamen, and spinal chord (data not shown). Several additional tissues not represented on the MTN blot were also found to contain cyclophilin F mRNA: paracentral gyrus, pons, corpus callosum, amygdala, caudate nucleus, hippocampus, and thalamus. Although not in the brain, the tissue that seemed to have the highest expression on the entire blot was the thyroid gland. These results are exciting because they suggest cyclophilin F is expressed in regions of the brain known to be affected by AD: the temporal lobe, hippocampus, and the amygdala (Citron, M., Diehl, T. S., Capell, A., Haass, C., Teplow, D. B., Selkoe, D. Neuron. 1996. 17:171-179.). It is quite possible that an overexpression of cyclophilin F in these regions of the brain could contribute in some way to the cause of AD by increasing Aβ levels. If cyclophilin F is overexpressed in these regions, then it is likely there would be an increase in Aβ production leading to an increase in amyloid plaque formation.

### Example 4

### 2268 Functional Gene Screen

The 2268 gene screen is performed in a slightly different manner than the 479 10 q screen in that an internal transfection control is used to normalize the data. In the 10 q screen, the results are represented as raw data, therefore cell death and level of expression are not controlled for because it is a technical impossibility. But in the larger screen there are many more data points making it necessary to control for transfection efficiency in order to produce data that is normally distributed. Since the ELISA experiment only uses the supernatants of the cells, the cells are used to read luciferase values to determine the level of transfection efficiency based on the luciferase signal. The luciferase plasmid is included in the original transfection mixture and read at 24 hours posttransfection. The results of the Aβ40 and Aβ42 ELISA screen are interpreted as the ratio of ELISA light units/luciferase light units and a high signal represents a greater ELISA signal/luciferase signal ratio and is interpreted as an inducer of Aβ40 or Aβ42.

The results of the Aβ40 and Aβ42 ELISA screens are put into a proprietary database for analysis; by so doing the results can be queried and presented in multiple formats. Table 2 shows the genes that were increased ≧5-fold in the Aβ40 and Aβ42 assay At first glance of the table, there is no specific type of gene that stands our from the rest. It seems as though many types of genes can increase Aβ40 and Aβ42 secretion compared to the vector control.

Genes that specifically increased Aβ40 secretion ≧1.5 fold with no affect on Aβ42 levels is shown in Table 3. This set of genes was not normalized to the luciferase control and was named the “raw” data. None of these genes overlapped with the normalized set of genes. We followed up with “raw” data hits because it is important to follow up with all potential Aβ modifiers, independent of how the modifier was discovered.

Only two genes were found to increase Aβ42 levels specifically in the “raw” data set. (See Table 4). These genes were also not found in the normalized data set. Therefore we picked the top 14 hits from the Aβ40 and Aβ42 ELISA to perform follow up validation experiments. All the genes from this screen are likely to be interesting AD targets because of the unique feature that they affect Aβ40 or Aβ42 secretion.

The raw data from each screen is analyzed to compare to the normalized data set. This internal comparison allowed the determination of the validity of this screening approach to find Aβ modifiers. Since there is little overlap between the raw data and the normalized data sets we are able to use follow up assays to empirically determine whether normalization is a better approach than reading raw data alone. Thus, our primary ELISA screens are followed up with clone retrieval and validation to confirm that the original ‘hits’ are real.

### Example 5

### Clone Retrieval and Validation

The top 17 hits from these assays are picked from their respective “mother plates” and fresh DNA isolated. Each clone was 5′ sequenced and the annotations confirmed or changed according to the sequence result. Subsequently, each clone was retransformed and the ELISA experiments were repeated, both for Aβ40 & Aβ42. These results are summarized in Table 10. Only 3/17 hits tested failed to repeat (18%), while the other 14 genes confirmed their activity.

To validate the hit's affect on Aβ levels, Western blots are performed. In the 14 genes tested, all confirmed their affect on increasing Aβ levels (data not shown). This result validates the ELISA results and provides the first evidence that these hits are novel candidate genes in the APP pathway because they can increase Aβ secretion.

At this point, HEK 293 cells are used instead of CHO K1 cells for the APP metabolism experiments (Western blots of the APP cleavage products). While the CHO K1 cells were a good cell line to use for the ELISA screen because of the low level of endogenous Aβ, endogenous APP processing activity in HEK 293 cells is quite high so it is convenient to measure APP cleavage products in these cells. In addition to measuring Aβ, Western blots are also used to examine APP and the APP metabolic products C99, C89, C83, sAPPα, and sAPPβ (data not shown) As it is likely that the increase in Aβ levels seen in the ELISA are due to an increase in BACE activity, alterations in cleavage products were assayed. Measuring the downstream products of BACE cleavage can be used to test for changes in activity. The sAPPα fragment was also measured to determine if the hits are having an affect on the α-secretase activity.

The effect of several “hits” on APP and CTF (C99, C89, & C83) levels were examined. Using the APPC8 antibody we were able to detect APP and CTFs on the same gel. RIPA cell extracts are loaded on 16% Tricine gels, resolved, blotted onto a PVDF membrane, and probed with APPC8. Co-transfection of BACE with APPwt and APPswe served as the positive control for the assay. BACE overexpression caused a decrease in total APP levels with the APPwt and APPswe substrates. Additionally, the levels of the C83 fragment were decreased while the levels of the C89 and C99 fragments were increased as compared to the vector transfected or APP transfected cell controls. The transfected HEK cells have about 5-fold more APP and APP cleavage products than the cells alone (data not shown). This is representative of the overexpression of the APP plasmid.

There are several hits that seemed to alter APP and CTF metabolism: Carboxypeptidase Z (CPZ), Protease E, CPBP, TOB3 (8I21), & Unknown (10M13) all decreased total APP and total CTF levels when co-transfected with APPwt. TOB3, Calmodulin 2, Carboxypeptidase Z, cyclophilin F, and TNFR aminopeptidase (8C8) both decreased APP and CTF levels when cotransfected with APPswe. The genes CTRB1, ANG2, and TNFR had no affect on APP or CTF levels when co-transfected with APPwt. Despite this wide variety of affects, the modulation of APP and CTF levels is a clear indication that the overexpression of these genes is directly affecting the cleavage of APP resulting in an increase in Aβ secretion.

sAPPα processing was also investigated by cotransfection of APPwt or APPswe and the cDNA followed by Western blots of the supernants. Data indicate that in the BACE transfected cells, sAPPα levels are very low in the APPwt samples and nonexistent in the APPswe samples. This is to be expected since the overexpression of BACE will compete the APP substrate away from the α-secretase enzyme. The levels of sAPPα in the HEK 293 cells alone is below the level of detection while the vector/APP transformed cells show considerable levels of sAPPα (data not shown). This indicates the endogenous α-secretase is active in the presence of overexpressed APP. In the APPwt condition, only Carboxypeptidase Z (CPZ) decreased sAPPα levels compared to the vector control. When co-transfected with APPswe, tryptase beta (10M11), TOB3 (8I21), Carboxypeptidase Z (CPZ), Calmodulin 2 (Cal2), Cyclophilin F, & TNFR aminopeptidase (8C8) decreased sAPPα to levels similar to the BACE transfection. These results suggest that the hits can mimic BACE-like activity(s) by increasing Aβ secretion and decreasing sAPPα secretion. This indicates the genes can stimulate the amyloidgenic β-site cleavage and decrease the nonamyloidgenic α-site cleavage of APP.

Our collection of Western blot results suggest the hits are specifically affecting the cleavage of APP through increases in BACE and/or activity. Therefore, we decided to provide the C99 substrate, rather than the APPwt substrate to the hits. In this way, we could directly assay the cleavage of C99 into Aβ40 and/or Aβ42 and determine whether the hits specifically increase GACE activity. Data from the 24 hr transient transfection of C99 in HEK 293 cells co-transfected with each ELISA hit in a 1:3 ratio indicate that C99 is processed by the hits resulting in altered levels of C99. The tryptase beta, TOB3, protease E, CPBP, BMP, ANG2, TNFR, CTRB1, and TLL2 clones all decreased C99 levels compared to the C99 control. In addition, the supernantants were assayed in the Aβ40 ELISA. Consistent with the increased processing of the C99 fragment shown in the Western blots, Aβ40 levels are increased in all the hits except for the aminohydrolase and CPBP genes. These results suggest that the hits can stimulate the cleavage of C99 into Aβ, arguing these hits are directly affecting γ-secretase activity or are novel γ-secretase activities themselves.

1. A method to treat, prevent or ameliorate pathological conditions associated with Aβ secretion comprising administering to a subject in need thereof an effective amount of one or more modulators of one or more proteins selected from the group consisting of those disclosed in Table 1.

2. The method of claim 1 wherein said proteins are selected from the group consisting of cyclophilin F, carboxypeptidase Z, Calmodulin 2, TOB3, TLL2, protease E, chymotrypsinogen B1, Bone morphorgenic protein BMP45 (BMP45), ANG2 gene, type 1 tumor necrosis factor receptor shedding aminopeptidase regulator (ARTS-1), Fas (TNFRSF6)-associated via death domain (FADD), mitogen-activated protein kinase 4 (MAPK4), N-acylsphingosine amidohydrolase (acid ceramidase) and tryptase beta.

3. The method of claim 1 wherein said modulator inhibits activity of said protein in said subject.

4. The method of claim 1 wherein said modulator inhibits gene expression of said protein in said subject.

5. The method of claim 1 wherein said modulator comprises any one or more substances selected from the group consisting of antisense oligonucleotides, triple helix DNA, ribozymes, RNA aptamers, siRNA and double or single stranded RNA wherein said substances are designed to inhibit gene expression of said protein.

6. The method of claim 1 wherein said modulator comprises one or more antibodies to said protein, or fragments thereof, wherein said antibodies or fragments thereof can inhibit activity of said protein.

7. The method of claim 1 wherein said pathological conditions are selected from the group consisting of Alzheimer's Disease, Parkinson's Disease, tauopathies, prion diseases, frontotemporal dementia, striatonigral degeneration, Lewd body dementia, Huntington's disease, Pick's disease, amyloidosis, and other neurodegenerative disorders associated with excess Aβ production.

8. A method to treat, prevent or ameliorate pathological conditions associated with Aβ secretion comprising administering to a subject in need thereof a pharmaceutical composition comprising an effective amount of one or more modulators of any one or more proteins selected from the group consisting of those disclosed in Table 1.

9. The method of claim 8 wherein said proteins are selected from the group consisting of cyclophilin F, carboxypeptidase Z, Calmodulin 2, TOB3, TLL2, protease E, chymotrypsinogen B1, Bone morphorgenic protein BMP45 (BMP45), ANG2 gene, type 1 tumor necrosis factor receptor shedding aminopeptidase regulator (ARTS-1), Fas (TNFRSF6)-associated via death domain (FADD), mitogen-activated protein kinase 4 (MAPK4), N-acylsphingosine amidohydrolase (acid ceramidase)and tryptase beta.

10. The method of claim 8 wherein said modulator inhibits activity of said protein in said subject.

11. The method of claim 8 wherein said modulator inhibits gene expression of said protein in said subject.

12. The method of claim 8 wherein said modulator comprises any one or more substances selected from the group consisting of antisense oligonucleotides, triple helix DNA, ribozymes, RNA aptamers, si RNA and double or single stranded RNA wherein said substances are designed to inhibit gene expression of said protein.

13. The method of claim 8 wherein said modulator comprises one or more antibodies to said protein, or fragments thereof, wherein said antibodies or fragments thereof can inhibit activity of said protein.

14. The method of claim 8 wherein said pathological conditions are selected from the group consisting of Alzheimer's Disease, Parkinson's Disease, tauopathies, prion diseases, frontotemporal dementia, striatonigral degeneration, Lewd body dementia, Huntington's disease, Pick's disease, amyloidosis, and other neurodegenerative disorders associated with excess Aβ production.

15. A method to identify modulators useful to treat, prevent or ameliorate pathological conditions associated with Aβ secretion comprising assaying for the ability of a candidate modulator to inhibit the activity of a protein selected from the group consisting of those disclosed in Table 1.

16. The method of claim 15 wherein said proteins are selected from the group consisting of cyclophilin F, carboxypeptidase Z, Calmodulin 2, TOB3, TLL2, protease E, chymotrypsinogen B1, Bone morphorgenic protein BMP45 (BMP45), ANG2 gene, type 1 tumor necrosis factor receptor shedding aminopeptidase regulator (ARTS-1), Fas (TNFRSF6)-associated via death domain (FADD), mitogen-activated protein kinase 4 (MAPK4), N-acylsphingosine amidohydrolase (acid ceramidase)and tryptase beta.

17. The method of claim 15 wherein said method further comprises assaying for the ability of an identified inhibitory modulator to reverse the pathological effects observed in any one or more animal models of said pathological conditions.

18. The method of claim 15 wherein said method further comprises assaying for the ability of an identified inhibitory modulator to reverse the pathological effects observed in clinical studies with subjects with said pathological conditions.

19. The method of claim 15 wherein said pathological conditions are selected from the group consisting Alzheimer's Disease, Parkinson's Disease, tauopathies, prion diseases, frontotemporal dementia, striatonigral degeneration, Lewd body dementia, Huntington's disease, Pick's disease, amyloidosis, and other neurodegenerative disorders associated with excess Aβ production.

20. A method to identify modulators useful to treat, prevent or ameliorate pathological conditions associated with Aβ secretion comprising assaying for the ability of a candidate modulator to inhibit gene expression of a protein selected from the group consisting of those disclosed on Table 1.

21. The method of claim 20 wherein said proteins are selected from the group consisting of cyclophilin F, carboxypeptidase Z, Calmodulin 2, TOB3, TLL2, protease E, chymotrypsinogen B1, Bone morphorgenic protein BMP45 (BMP45), ANG2 gene, type 1 tumor necrosis factor receptor shedding aminopeptidase regulator (ARTS-1), Fas (TNFRSF6)-associated via death domain (FADD), mitogen-activated protein kinase 4 (MAPK4), N-acylsphingosine amidohydrolase (acid ceramidase) and tryptase beta.

22. The method according to claim 20 wherein said method further comprises assaying for the ability of an identified inhibitory modulator to reverse the pathological effects observed in any one or more animal models of said pathological conditions.

23. The method according to claim 20 wherein said method further comprises assaying for the ability of an identified inhibitory modulator to reverse the pathological effects observed in clinical studies with subjects with said pathological conditions.

24. The method of claim 20 wherein said pathological conditions are selected from the group consisting of Alzheimer's Disease, Parkinson's Disease, tauopathies, prion diseases, frontotemporal dementia, striatonigral degeneration, Lewd body dementia, Huntington's disease, Pick's disease, amyloidosis, and other neurodegenerative disorders associated with excess Aβ production.

25. A pharmaceutical composition comprising one or more modulators of any one or more proteins selected from the group consisting of those disclosed in Table 1 in an amount effective to treat or ameliorate pathological conditions associated with Aβ secretion in a subject in need thereof.

26. The pharmaceutical composition according to claim 25 wherein said proteins are selected from the group consisting of cyclophilin F, carboxypeptidase Z, Calmodulin 2, TOB3, TLL2, protease E, chymotrypsinogen B1, Bone morphorgenic protein BMP45 (BMP45), ANG2 gene, type 1 tumor necrosis factor receptor shedding aminopeptidase regulator (ARTS-1), Fas (TNFRSF6)-associated via death domain (FADD), mitogen-activated protein kinase 4 (MAPK4), N-acylsphingosine amidohydrolase (acid ceramidase) and tryptase beta.

27. The pharmaceutical composition according to claim 25 wherein said pathological conditions are selected from the group consisting of Alzheimer's Disease, Parkinson's Disease, tauopathies, prion diseases, frontotemporal dementia, striatonigral degeneration, Lewd body dementia, Huntington's disease, Pick's disease, amyloidosis, and other neurodegenerative disorders associated with excess Aβ production.

28. The pharmaceutical composition according to claim 25 wherein said modulator inhibits the activity of said protein.

29. The pharmaceutical composition according to claim 25 wherein said modulator inhibits gene expression of said protein.

30. The pharmaceutical composition of claim 25 wherein said modulator comprises any one or more substances selected from the group consisting of antisense oligonucleotides, triple helix DNA, ribozymes, RNA aptamer, siRNA and double or single stranded RNA wherein said substances are designed to inhibit gene expression of said protein.

31. The pharmaceutical composition of claim 25 wherein said modulator comprises one or more antibodies to said protein, or fragments thereof, wherein said antibodies or fragments thereof can inhibit activity of said protein.

32. A method to diagnose subjects suffering from pathological conditions associated with Aβ secretion who may be suitable candidates for treatment with one or more modulators of any one or more proteins selected from the group consisting of those disclosed in Table 1 comprising assaying mRNA levels of said protein in a biological sample from said subject wherein a subject with increased mRNA levels compared to controls would be a suitable candidate for modulator treatment.

33. The method of claim 32 wherein said proteins are selected from the group consisting cyclophilin F, carboxypeptidase Z, Calmodulin 2, TOB3, TLL2, protease E, chymotrypsinogen B1, Bone morphorgenic protein BMP45 (BMP45), ANG2 gene, type 1 tumor necrosis factor receptor shedding aminopeptidase regulator (ARTS-1), Fas (TNFRSF6)-associated via death domain (FADD), mitogen-activated protein kinase 4 (MAPK4), N-acylsphingosine amidohydrolase (acid ceramidase) and tryptase beta.

34. A method to treat, prevent or ameliorate pathological conditions associated with Aβ secretion comprising
(a) assaying a subject for mRNA levels for any one or more proteins selected from the group consisting of those disclosed in Table 1; and,
(b) administering to a subject with increased levels of mRNA compared to controls a modulator of any one or more of said proteins in an amount sufficient to treat or ameliorate said pathological conditions.

35. The method of claim 34 wherein said proteins are selected from the group consisting of cyclophilin F, carboxypeptidase Z, Calmodulin 2, TOB3, TLL2, protease E, chymotrypsinogen B1, Bone morphorgenic protein BMP45 (BMP45), ANG2 gene, type 1 tumor necrosis factor receptor shedding aminopeptidase regulator (ARTS-1), Fas (TNFRSF6)-associated via death domain (FADD), mitogen-activated protein kinase 4 (MAPK4), N-acylsphingosine amidohydrolase (acid ceramidase)and tryptase beta.

36. The method of claim 33 wherein said pathological conditions are selected from the group consisting of Alzheimer's Disease, Parkinson's Disease, tauopathies, prion diseases, frontotemporal dementia, striatonigral degeneration, Lewd body dementia, Huntington's disease, Pick's disease, amyloidosis, and other neurodegenerative disorders associated with excess Aβ production.

37. A method to treat, prevent or ameliorate pathological conditions associated with Aβ secretion comprising:
(a) assaying a subject for protein levels of any one or more proteins selected from the group consisting of those disclosed in Table 1; and,
(b) administering to a subject with increased protein levels compared to controls a modulator of any one or more of said proteins in an amount sufficient to treat or ameliorate said pathological conditions.

38. The method of claim 37 wherein said proteins are selected from the group consisting of cyclophilin F, carboxypeptidase Z, Calmodulin 2, TOB3, TLL2, protease E, chymotrypsinogen B1, Bone morphorgenic protein BMP45 (BMP45), ANG2 gene, type 1 tumor necrosis factor receptor shedding aminopeptidase regulator (ARTS-1), Fas (TNFRSF6)-associated via death domain (FADD), mitogen-activated protein kinase 4 (MAPK4), N-acylsphingosine amidohydrolase (acid ceramidase)and tryptase beta.

39. The method of claim 37 wherein said pathological conditions are selected from the group consisting of Alzheimer's Disease, Parkinson's Disease, tauopathies, prion diseases, frontotemporal dementia, striatonigral degeneration, Lewd body dementia, Huntington's disease, Pick's disease, amyloidosis, and other neurodegenerative disorders associated with excess Aβ production.

40. A diagnostic kit for detecting mRNA levels or protein levels of a protein selected from the group consisting of those disclosed in Table 1 in a biological sample, said kit comprising:
(a) a polynucleotide of said protein or a fragment thereof;
(b) a nucleotide sequence complementary to that of (a);
(c) an RNAi sequence complementary to that of (a);
(d) said protein, or a fragment thereof; or
(e) an antibody to said protein wherein components (a), (b), (c), (d) or (e) may comprise a substantial component.
